(19) United States
(12) Patent Application Publication
(10) Pub. No.: US 2024/0342673 A1
(43) Pub. Date: Oct. 17, 2024

(54) SYSTEMS AND METHODS FOR PROVIDING OPTIMAL PROCESS PARAMETERS FOR FLUID BED GRANULATION SYSTEMS

(71) Applicant: R.P. Scherer Technologies, LLC, Carson City, NV (US)

(72) Inventors: Amin ABEDINI, Lexington, KY (US); Christin HOLLIS, Paris, KY (US)

(73) Assignee: R.P. Scherer Technologies, LLC, Carson City, NV (US)

(21) Appl. No.: 18/300,905

(22) Filed: Apr. 14, 2023

(51) Int. Cl. B01J 2/16; G06F 30/28

(52) U.S. Cl. CPC B01J 2/16; G06F 30/28; G06F 2113/08

(57) ABSTRACT

A user interface (UI) for providing optimized parameters for a fluid bed granulation process may include a first user input field for receiving intrinsic properties of an input powder, a second user input field for receiving granulation requirements for granules formed from the input powder during the fluid bed granulation process, a third user input field for receiving operational capabilities of a fluid bed granulation system, and an output field configured to display optimal process parameters for the fluid bed granulation system. The optimal process parameters may be determined by thermodynamically modeling granulation of the input powder in the fluid bed granulation system.

### FIELD

The present disclosure relates generally to fluid bed granulation processes, in particular to systems and methods for providing optimal process parameters for operating fluid bed granulation systems.

### BACKGROUND

Fluid bed granulation is an industrial process for creating granules from powders. In the pharmaceutical industry, fluid bed granulation is commonly employed to granulate active pharmaceutical ingredients with inactive substances that serve as a matrix for the active pharmaceutical ingredient (API). Granules produced using fluid bed granulation are homogeneous, highly dispersible, and highly compressible. As a result, the granules can be easily compressed, for example to form pharmaceutical tablets to be provided to patients. In addition, unlike other granulation processes, fluid bed granulation can be performed entirely within a single piece of equipment, minimizing the labor required to and losses incurred by transferring materials between multiple equipment units.

Using a fluid bed granulation process to produce granules of a desired size and density that meet product specifications requires precise selection of the values of several process parameters on the fluid bed granulation system. These values are typically estimated empirically or through trial-and-error by highly trained experts who have experience operating the particular granulation system that is being used to for the fluid bed granulation process. Since the raw materials (e.g., the API) are often costly and may be available in limited quantities, estimating the optimal values for the process parameters using empirical trial-and-error practices may increase research and development costs and can cause delays in product manufacturing timelines.

### SUMMARY

As discussed, fluid bed granulation processes can produce granules with beneficial properties, including high dispersibility and high compressibility. However, relying on expert fluid bed granulation system operators to estimate the values of the process parameters necessary to produce proper granules is restrictive, inefficient, and non-quantifiable. Gaining the experience required to accurately estimate the appropriate values of the process parameters can take many years, since the values of said parameters depend uniquely on the input powder being granulated, the make and model of the granulation system being used, and the desired characteristics of the granules to be produced. Furthermore, such practices require executing multiple trial-and-error experiments, which can significantly increase research and development cost and time.

Described are systems and methods for providing optimal process parameters for fluid bed granulation systems. A user interface (e.g., a graphical user interface displayed on a laptop or a smart phone) may be used to receive information from a user relevant to a fluid bed granulation process to be performed, including information about the input powder (e.g., material characteristics of the input powder), information about the desired product (e.g., drug product design specifications such as desired critical quality attributes (CQAs)), and information about the operational capabilities of the fluid bed granulation system being used for the granulation process. After the necessary information has been provided, a computer system connected to the user interface may use the information input by the user to model the thermodynamics of the granulation of the input raw materials in the granulation system. Optimal process parameters tailored to both the input powder and the granulation system may be determined using the thermodynamic model as well as historical databases and subsequently displayed on the user interface. The user can then set the setpoints of the process parameters for the fluid bed granulation system by simply entering the optimal values provided on the user interface into the granulation system.

The process of receiving the information necessary to determine optimal values for process parameters of fluid bed granulation systems may be streamlined by the user interface. Highly organized user input fields may be displayed to inform users of the specific information that they need to provide. Any user with access to the information—which is usually available from manufacturers of the input raw materials and the fluid bed granulation system—can input the information into the interface. To ensure that users enter information properly (e.g., in the proper format or with the proper units), the user interface may be configured to display additional details about the information upon user request. If an issue with a particular user input is detected, the interface may display a warning to the user that prompts the user to verify the accuracy of their input before the optimal process parameters are displayed. This may allow even users who are inexperienced with fluid bed granulation to successfully operate fluid bed granulation systems.

In addition to reducing reliance on experts, the described systems and methods may significantly reduce—or, in many cases, remove entirely—the need for feasibility or trial-and-error granulation batches. The determination of the optimal process parameters by the computing system using the thermodynamic model of the granulation of input raw materials may be highly systematic and based upon well-tested physical laws. This may reduce the influence of human error and, as a result, may increase research and development efficiency.

A method for providing optimal process parameters for a fluid bed granulation process may comprise receiving from a user a plurality of intrinsic properties of an input powder, wherein the plurality of intrinsic properties comprises a bulk density of particles of the input powder and a threshold temperature value or a temperature range associated with degradation of an attribute of the input powder, receiving from the user granulation requirements for granules formed from the input powder during the fluid bed granulation process, wherein the granulation requirements comprise a granule size distribution and a required granule density, receiving from the user a plurality of operational capabilities of a fluid bed granulation system, wherein the plurality of operational capabilities of the fluid bed granulation system comprises a minimum and a maximum volume capacity, a spray rate range, an inlet air flow range, an inlet air temperature range, and an inlet air dew point range, thermodynamically modeling, using the plurality of intrinsic properties of the input powder, the granulation requirements, and the plurality of operational capabilities of the fluid bed granulation system, granulation of the input powder in the fluid bed granulation system to determine optimal process parameters for the fluid bed granulation system, wherein the optimal process parameters comprise an inlet air temperature, an inlet air dew point, and an inlet air flow rate of air supplied into an inlet of the fluid bed granulation system, and providing the optimal process parameters for the fluid bed granulation system on a user interface.

In some embodiments of the method, thermodynamically modeling granulation of the input powder in the fluid bed granulation system comprises determining an absolute humidity of exhaust air expelled by the fluid bed granulation system using the threshold temperature value or the temperature range associated with degradation of the attribute of the input powder.

In some embodiments of the method, thermodynamically modeling granulation of the input powder in the fluid bed granulation system comprises determining a specific enthalpy of exhaust air expelled by the fluid bed granulation system using the threshold temperature value or the temperature range associated with degradation of the attribute of the input powder and the absolute humidity of exhaust air.

In some embodiments of the method, thermodynamically modeling granulation of the input powder in the fluid bed granulation system comprises determining a specific enthalpy air at the inlet to the fluid bed granulation system based on the specific enthalpy of exhaust air expelled by the fluid bed granulation system.

In some embodiments of the method, the inlet air temperature is determined based on the specific enthalpy and dew point of the air at the inlet to the fluid bed granulation system.

In some embodiments of the method, thermodynamically modeling granulation of the input powder in the fluid bed granulation system comprises determining an absolute humidity of air at the inlet to the fluid bed granulation system using the inlet air temperature and enthalpy.

In some embodiments of the method, thermodynamically modeling granulation of the input powder in the fluid bed granulation system comprises determining a wet-bulb temperature of air at the inlet to the fluid bed granulation system using temperature and the absolute humidity of air at the inlet to the fluid bed granulation system.

In some embodiments of the method, thermodynamically modeling granulation of the input powder in the fluid bed granulation system comprises determining a drying capacity of the fluid bed granulation system based on the absolute humidity of the inlet air at the dry-bulb temperature and at the wet-bulb temperature.

In some embodiments of the method, thermodynamically modeling granulation of the input powder in the fluid bed granulation system comprises determining a drying rate of the fluid bed granulation system based on the absolute humidity of the inlet air at the dry bulb temperature and the absolute humidity of the exhaust air at the dry-bulb temperature.

In some embodiments of the method, the inlet air flow rate of air supplied into an inlet of the fluid bed granulation system is determined based on the drying rate and spray rate for the fluid bed granulation system.

In some embodiments, the method comprises providing the optimal process parameters for the fluid bed granulation system to the fluid bed granulation system and granulating the input powder using the fluid bed granulation system.

A user interface for providing optimal process parameters for a fluid bed granulation process may comprise a first input field for receiving, from a user, a plurality of intrinsic properties of an input powder, wherein the plurality of intrinsic properties comprises a bulk density of particles of the input powder and a threshold temperature value or a temperature range associated with degradation of an attribute of the input powder, a second input field for receiving, from the user, granulation requirements for granules formed from the input powder during the fluid bed granulation process, wherein the granulation requirements comprise a granule size distribution and a required granule density, a third input field for receiving, from the user, a plurality of operational capabilities of a fluid bed granulation system, wherein the plurality of operational capabilities of the fluid bed granulation system comprises a minimum and a maximum volume capacity, a spray rate range, an inlet air flow range, an inlet air temperature range, and an inlet air dew point range, and an output field configured to provide optimal process parameters for the fluid bed granulation system, wherein the optimal process parameters are determined by thermodynamically modeling, using the plurality of intrinsic properties of the input powder, the granulation requirements, and the plurality of operational capabilities of the fluid bed granulation system, granulation of the input powder in the fluid bed granulation system, and wherein the optimal process parameters comprise an inlet air temperature, an inlet air dew point, and an inlet air flow rate for air supplied into an inlet of the fluid bed granulation system.

In some embodiments, the user interface is configured to display an information window comprising information associated with the first user input field, the second user input field, or the third user input field upon user request.

In some embodiments, the information associated with the first user input field, the second user input field, or the third user input field comprises information about a format of a requested user input or a description of a requested user input.

In some embodiments, the user interface is configured to display a warning window indicating that a possible error has been detected when the user provides a value to the first user input field, the second user input field, or the third user input field that is outside of a predetermined range.

In some embodiments, the output field provides the optimal process parameters by displaying values of the optimal process parameters.

In some embodiments, the output field provides the optimal process parameters by providing a downloadable file comprising the optimal process parameters.

In some embodiments, the user interface is configured to provide information about inputting the optimal process parameters into the fluid bed granulation system upon user request.

A system for providing optimal process parameters for a fluid bed granulation process may comprise a fluid bed granulation system, a user interface comprising: a first input field configured to receive, from a user, a plurality of intrinsic properties of an input powder, wherein the plurality of intrinsic properties comprises a bulk density of particles of the input powder and a threshold temperature value or a temperature range associated with degradation of an attribute of the input powder, a second input field configured to receive, from the user, granulation requirements for granules formed from the input powder during the fluid bed granulation process, wherein the granulation requirements comprise a granule size distribution and a required granule density, a third input field configured to receive, from the user, a plurality of operational capabilities of a fluid bed granulation system, wherein the plurality of operational capabilities of the fluid bed granulation system comprises a minimum and a maximum volume capacity, a spray rate range, an inlet air flow range, an inlet air temperature range, and an inlet air dew point range, and an output field, and a computing system comprising one or more memories and one or more processors configured to: receive, from the user interface, the plurality of intrinsic properties of the input powder, the granulation requirements, and the plurality of operational capabilities of the fluid bed granulation system, thermodynamically model, using the plurality of intrinsic properties of the input powder, the granulation requirements, and the plurality of operational capabilities of the fluid bed granulation system, granulation of the input powder in the fluid bed granulation system to determine optimal process parameters for the fluid bed granulation system, wherein the optimal process parameters comprise an inlet air temperature, an inlet air dew point, and an inlet air flow rate for air supplied into an inlet of the fluid bed granulation system, and provide, using the output field of the user interface, the optimal process parameters for the fluid bed granulation system.

### BRIEF DESCRIPTION OF THE FIGURES

Various aspects of the disclosed systems and methods are set forth with particularity in the appended claims. A better understanding of the features and advantages of the disclosed systems and methods can be obtained by reference to the detailed description of illustrative embodiments and the accompanying drawings.

FIG. 1 shows a system for providing optimal process parameters for a fluid bed granulation system, according to some embodiments.

FIG. 2 shows an exemplary fluid bed granulation system, according to some embodiments.

FIG. 3 shows a user interface for providing optimal process parameters for a fluid bed granulation system, according to some embodiments.

FIG. 4 shows a method for providing optimal process parameters for a fluid bed granulation system, according to some embodiments.

FIG. 5 shows a method for thermodynamically modelling granulation of an input powder in a fluid bed granulation system, according to some embodiments.

FIG. 6A shows a user interface for providing optimal process parameters for a fluid bed granulation system prior to receiving user inputs, according to some embodiments.

FIG. 6B shows a user input information window displayed on a user interface for providing optimal process parameters for a fluid bed granulation system, according to some embodiments.

FIG. 6C shows an example user input field on a user interface for providing optimal process parameters for a fluid bed granulation system, according to some embodiments.

FIG. 6D shows an example user input field on a user interface for providing optimal process parameters for a fluid bed granulation system, according to some embodiments.

FIG. 6E shows an example input error window displayed on a user interface for providing optimal process parameters for a fluid bed granulation system, according to some embodiments.

FIG. 6F shows an example user control on a user interface for providing optimal process parameters for a fluid bed granulation system, according to some embodiments.

FIG. 6G shows an example output information window displayed on a user interface for providing optimal process parameters for a fluid bed granulation system, according to some embodiments.

FIG. 7 shows a method for granulating an input powder using optimal process parameters provided by a user interface for providing optimal process parameters for a fluid bed granulation system, according to some embodiments.

FIG. 8 shows exemplary relationships between thermodynamic quantities that can be used to thermodynamically model granulation of an input powder in a fluid bed granulation system, according to some embodiments.

### DETAILED DESCRIPTION

Fluid bed granulation is widely used to form granules from powdered materials—for example, a powdered active pharmaceutical ingredient (API) mixed with various inactive ingredients. During a fluid bed granulation process, input powder is heated and fluidized within a chamber of a fluid bed granulation system. The fluidized powder is sprayed with a binder solution to cause the powder to agglomerate and form granules, which are then dried to a particular moisture level before being discharged from the granulation system for further processing (e.g., to be compressed into tablets or to be encapsulated).

One major advantage of fluid bed granulation over other granulation processes is the reliance on only a single piece of equipment: the fluid bed granulation system. There exist a variety of fluid bed granulation systems, each of which have unique properties that can impact the granulation process and, consequently, the granules that are produced. The values of some of these properties are adjustable and must be specified by an operator of the fluid bed granulation system in order for the granulation to be performed. These adjustable properties, referred to herein as the “process parameters” of fluid bed granulation systems, can include parameters such as the spray rate of the binder solution and the temperature, dew point, and flow rate of the process air that is supplied through the inlet of the granulation system to heat and fluidize the input powder.

Conventionally, highly trained experts are employed to estimate the values of the adjustable parameters of a fluid bed granulation system prior to the initiation of a fluid bed granulation process. However, reliance on experts is expensive and inefficient, since these individuals may require extensive training and years of experience before they are capable of accurately estimating the process parameter values needed to granulate a given input powder. Furthermore, human estimates are prone to human error. Incorrect estimates of process parameter values can severely damage the input powder, rendering it unusable once granulated. Typically, empirical trial-and-error experiments are executed before finalizing the process parameters; such trial-and-error practices, however, can be both expensive and time-consuming.

Accordingly, described are systems and methods for providing operators of fluid bed granulation systems with optimal process parameters for said fluid bed granulation systems. The systems and methods leverage a user interface (UI) to allow an operator to easily input information about the input raw materials (referred to hereinafter as “input powder”) to be granulated, the desired properties of the granules to be produced, and the fluid bed granulation system being used to execute the granulation process. The UI may provide guidance to the operator as they input the information, for example by displaying information about the information being requested or by warning the user when a possible error has been detected. This may be particularly useful for operators inexperienced with fluid bed granulation or operators unfamiliar with the particular input powder being granulated or granulation system being used.

The user interface may be connected to a computer system. After the user has input the necessary information, the computing system may use the information to thermodynamically model the granulation of the input powder. Thermodynamically modeling the granulation process may involve determining the values of various thermodynamic properties (e.g., temperature and humidity) at different locations within the fluid bed granulation system or at different stages of the fluid bed granulation process based upon characteristics of the input powder (e.g., temperatures to which the powder can be safely exposed without risking degradation) and characteristics of the granulation system (e.g., the maximum rate at which the system can spray the binder solution). The computing system may then determine optimal values for the process parameters of the fluid bed granulation system using the thermodynamic model. By systematically determining the optimal process parameters using well-tested physical laws implemented on a computing system rather than estimating the parameter values based on experience, the provided systems and methods allow non-expert users to successfully operate fluid bed granulation systems with minimal error or loss. As a result, research and development cost- and time-efficiency may significantly increase.

Once the optimal process parameters have been determined, the parameters may be automatically displayed on the UI. The UI may be configured to display or link to additional information about each process parameter upon user request or may provide users with instructions for setting the values of the process parameters for the fluid bed granulation system. In some cases, the UI can present users with options to save, share, or download the optimal process parameters, which may help with record-keeping or with increasing the overall efficiency of the granulation process.

### System

A system for providing optimal process parameters for a fluid bed granulation system may include a computing system, a user interface, and the fluid bed granulation system itself. The user interface may contain input fields configured to receive information about a fluid bed granulation process to be performed. After the necessary information has been entered into the user interface (e.g., by an operator of the fluid bed granulation system), the computing system, which may be communicatively coupled to the user interface, may determine optimal values for process parameters of the fluid bed granulation system. The computing system may then cause the user interface to display the optimal process parameters in an output field of the user interface.

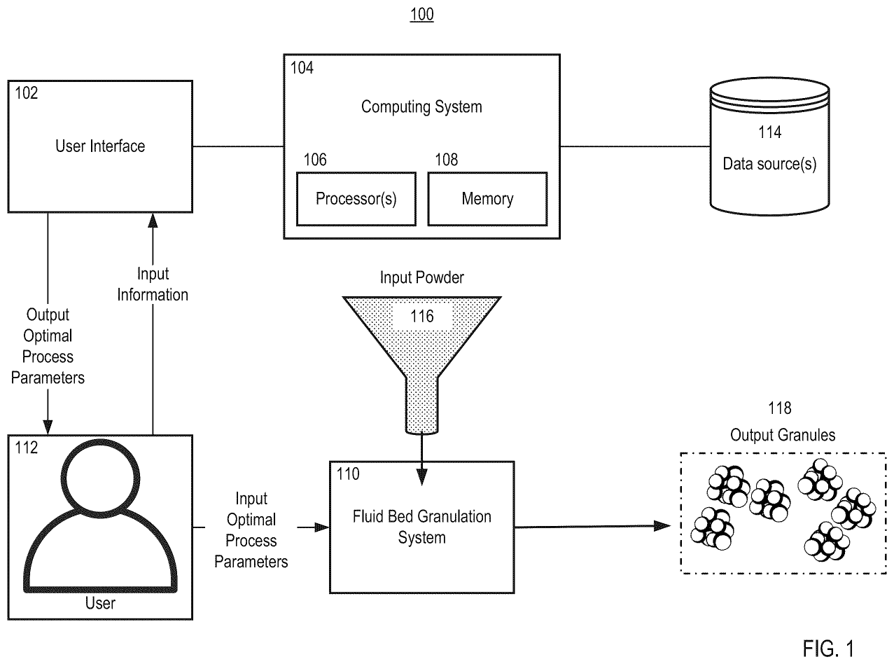

FIG. 1 shows an exemplary system 100 for providing optimal process parameters for a fluid bed granulation system 110. User 112, who may be an operator of fluid bed granulation system 110, may wish to use granulation system 110 to form granules 118 from an input powder 116. In order to do so, user 112 may need to set the values of one or more process parameters for fluid bed granulation system 110. Setting these values correctly may be critical to producing granules with desired characteristics such as particle size distribution and density.

To determine the appropriate values of the process parameters, user 112 may input information associated with the fluid bed granulation process to be performed into a user interface (UI) 102. UI 102 may be a graphical user interface (GUI), a command line interface, a menu-driven interface, a form-based interface, or a natural language interface. UI 102 may include a display, such as an LCD display, an LED display, a display for a personal computer, or a display for a mobile device (e.g., a touch screen for a smart phone). User 112 may interact with UI 102 using one or more user controls, for example a computer mouse, a keyboard, a microphone, or a touch pad (e.g., a touch pad for a laptop).

UI 102 may provide user 112 with one or more user input fields configured to receive the information about the granulation process, for example by displaying the input fields on the display. The user input fields may prompt user 112 to provide information about input powder 116, fluid bed granulation system 110, and requirements for granules 118 to be produced. If user 112 requires additional guidance or assistance, for example assistance identifying where the necessary information may be found or the format in which information should be provided, UI 102 may be configured to display (e.g., in a pop-up window) instructions or descriptions associated with the various user input fields.

Information input into UI 102 by user 112 may be received by a computing system 104 that is communicatively coupled to UI 102 (e.g., through a wired connection or a wireless connection). Computing system 104 may be any device or collection of devices that include at least one processor 106 and at least one memory 108. For example, computing system 104 may be or may include a desktop computer, a laptop computer, a tablet computer, a mobile device (e.g., a smart phone), or a server. Memory 108 may include any device configured to provide storage, including electrical, magnetic, or optical memory, for instance a random-access memory (RAM), a cache, a hard drive, a CD-ROM drive, a tape drive, or a removable storage disk. Memory 108 may store software comprising programs or instructions for executing methods for providing optimal process parameters for fluid bed granulation systems. In addition to receiving information from UI 102, computing system 104 may be configured to receive information from other data sources 114, such as other computers, other servers, databases, or other users.

Using the information received via UI 102, computing system 104 may be configured to thermodynamically model granulation of input powder 116 in fluid bed granulation system 110. Thermodynamically modeling granulation of input powder 116 may involve determining thermodynamic properties at various locations within granulation system 110 at various stages of the fluid bed granulation process to determine how input powder 116 will be affected by the fluid bed granulation process in fluid bed granulation system 110. This may allow computing system 104 to determine optimal values of the process parameters for fluid bed granulation system 110 that will allow granules 118 with the desired properties to be produced.

Once computing system 104 has determined the optimal values of the process parameters, processors 106 may cause user interface 102 to provide the optimal process parameters to user 112, for example by causing the display of UI 102 to display the optimal process parameters. Now having been provided with the optimal process parameters, user 112 may initiate the granulation of input powder 116 by inputting the optimal process parameters into fluid bed granulation system 110.

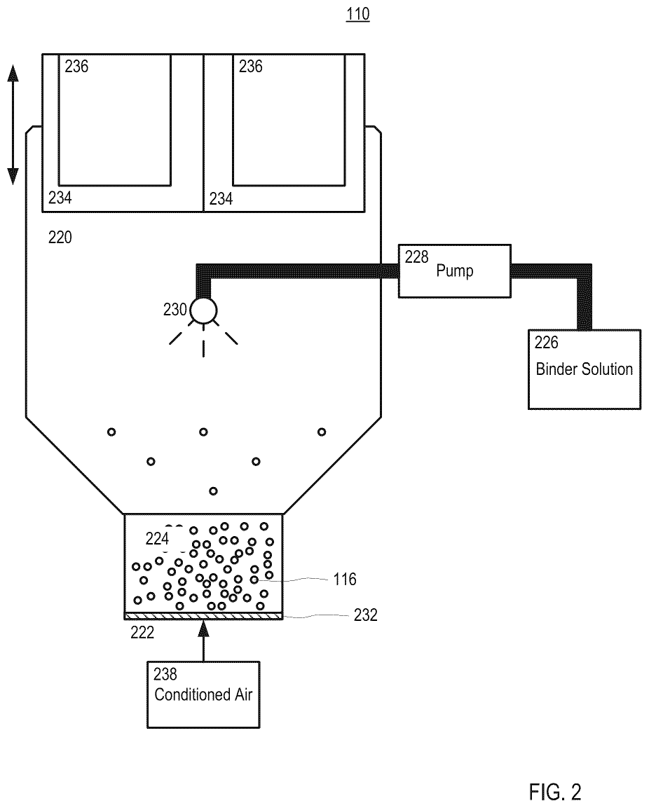

Fluid bed granulation system 110 may be configured in a top-spray, bottom-spray, or tangential fluid bed granulation system. For illustrative purposes, a top-spray fluid bed granulation system is shown in FIG. 2. In a top-spray processor, raw input powder 116 may be placed in a product container 224 on a retainer screen 232. The volume of input powder that container 224 is capable of receiving may depend on the make and model of the granulation system and may be one of the limiting factors that is used to determine the optimal process parameters for the granulation process.

The granulation process may begin when system 110 injects conditioned air 238 upwards through an inlet 222 and retainer screen 232 to container 224 to fluidize particles of input powder 116. The properties of conditioned air 238 at inlet 222 including temperature and dew point, along with the rate at which conditioned air 238 flows through inlet 222, may be among the process parameters for granulation system 110. The temperature of conditioned air 238 may impact the quality of the granules to be produced. If conditioned air 238 is too hot, important attributes of input powder 116 (e.g., the safety and/or efficacy of input powder 116) may degrade or be destroyed during the granulation process. The flow rate of conditioned air 238 through inlet 222 may also impact granule quality. If the flow rate is too high, granules of the desired size and density may not form; on the other hand, if the flow rate is too low, input powder 116 may not be sufficiently fluidized.

The fluidized particles of input powder 116 may be forced upwards and fluidized inside of an expansion chamber 220. Granulation system 110 may then pump a binder solution 226, which may be an aqueous solution of inactive or active ingredients, into expansion chamber via one or multiple nozzles 230 using a pump 228. Nozzle(s) 230 may spray binder solution 226 in a direction opposite to the flow of conditioned air 238. Moist air may be removed from expansion chamber via an exhaust system 234, which may comprise one or more filters 236 configured to separate the particles from the exhaust air. Filter(s) 236 may periodically shake in order to return particles separated from the exhaust air to the fluidized bed. The fluidized particles may adhere to liquid droplets of the binder solution and form granules. These granules may collect on retaining screen 232, where the fluidized bed is created. The fluid bed granulation process may continue until the entire quantity of input powder 116 has been agglomerated or until the entire quantity of binder solution 226 has been sprayed. Once spraying of binder solution 226 has ceased, conditioned air 238 may continue to be supplied through inlet 222 until the granules have reached a pre-determined moisture content.

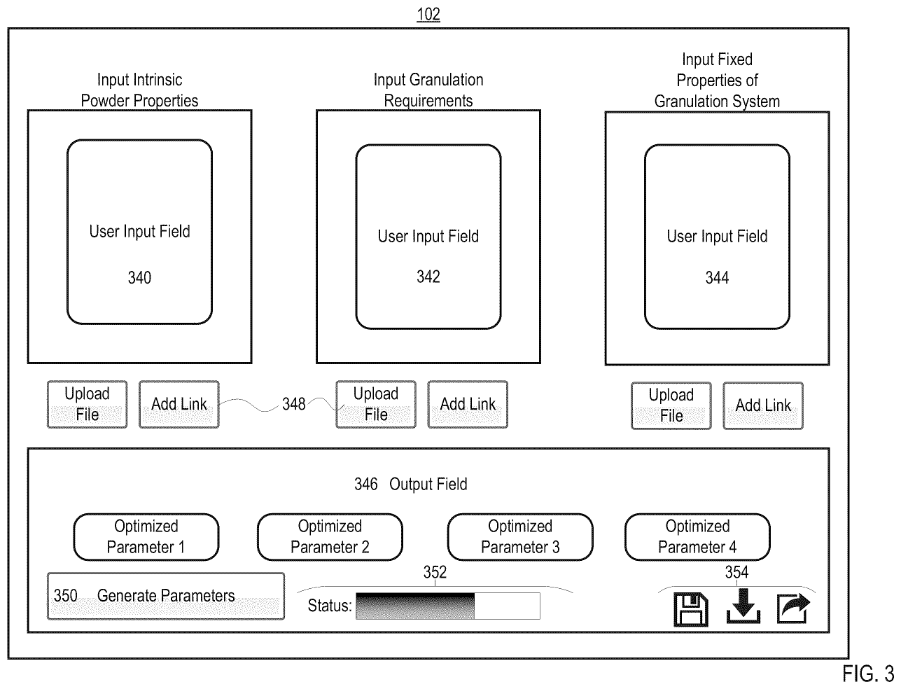

User interface 102 may provide optimal values of process parameters for a fluid bed granulation system (e.g., system 110) to operators of the system so that the operators do not have to rely on their own estimates. An exemplary implementation of UI 102 is illustrated in FIG. 3.

As shown, UI 102 may comprise a plurality of user input fields configured to receive information associated with a fluid bed granulation process to be performed that may affect the values of the process parameters for the fluid bed granulation system. The user input fields may comprise a first user input field 340 configured to receive information about the input powder, a second user input field 342 configured to receive information about granulation requirements, and a third user input field 344 configured to receive information about fixed (i.e., non-adjustable) properties of the fluid bed granulation system that will be used for the granulation of the input powder.

Each user input field may be configured to receive numerous forms of data, including text-based data, numerical data, image data, or sound-based data. Each user input field may be displayed in a single window (as shown in FIG. 3), in separate windows, or in separate tabs of a single window.

UI 102 may include one or more user input controls 348 (e.g., one or more selectable icons) that allow users to upload files related to the information being requested or add links (e.g., hyperlinks) related to the information being requested. In such cases, the computing system that controls UI 102 (e.g., computing system 104 shown in FIG. 1) may be configured to evaluate and automatically populate one or more of the user input fields using the uploaded file or added link.

The input powder properties configured to be received by user input field 340 may include physical properties of the input powder that may affect the granulation of the input powder. Such physical properties may include, for example, the bulk density of the input powder. User input field 340 may also be configured to prompt users to provide information about the temperature tolerance of the input powder, for example a threshold temperature value or a temperature range associated with degradation of an attribute (e.g., the efficacy and/or safety) of the input powder. The information about the input powder may be provided to the user by the manufacturer or developer of the input powder.

The granulation requirements configured to be received by user input field 342 may include physical properties that granules produced by the fluid bed granulation system should have. These granule properties may include a desired granule size distribution and a required granule density. The granulation requirements may be provided (for example) by a party planning to purchase the granules of input powder for further processing or distribution.

The fixed properties of the fluid bed granulation system configured to be received by user input field 344 may include any unchangeable physical properties of the granulation system that may impact the thermodynamics of the granulation process. Such properties may include a maximum rate at which the granulation system can spray liquid binder and a maximum volume of input powder that the granulation system can granulate during a given granulation session. In some cases, information about the specific makes and models of granulation systems being used by a particular operator may be stored in the memory of the computing system that controls UI 102, and the processors of the computing system may automatically populate user input field 344 upon prompting by the user (e.g., when the user selects a particular make and model of a granulation system from a stored list of granulation systems that is displayed in user input field 344).

After the user has entered the required information into the user input fields of UI 102, they may prompt the computing system to provide the optimal process parameters for the granulation system in an output field 346, for example using a user output control 350. The computing system may also be configured to automatically begin the process of providing the optimal process parameters as soon as the necessary information has been input by the user. UI 102 may be configured to display output field 346 in the same window as one or more of the user input fields (as shown in FIG. 3), in a separate window from the window in which the user input fields are displayed, or in a different tab of the same window in which the user input fields are displayed. As the computing system determines the optimal values of the process parameters, UI 102 may display the computing system's progress, for example using a visual status indicator 352.

Once the computing system has determined the optimal values of the process parameters of the fluid bed granulation system, UI 102 may provide the optimized values in output field 346. The values may be displayed (as shown in FIG. 3) or may be provided as a file to be downloaded, for example a spreadsheet file or a .CSV file. Output field 346 may include one or more user controls 354 configured to allow the user to save, download, print, or share the provided optimal process parameters to, for example, facilitate the user's record-keeping associated with the granulation process, or to allow the user to send the optimized parameters to another party.

### Method

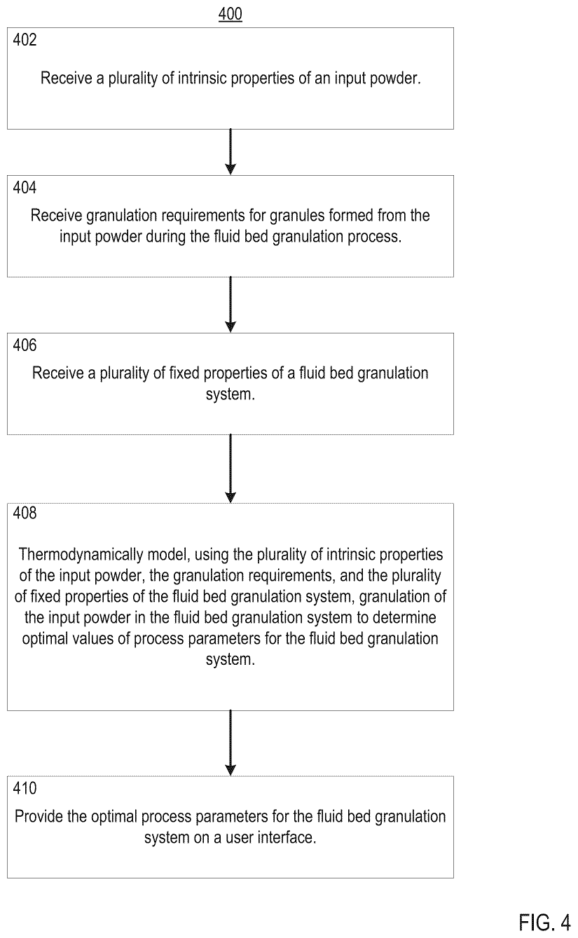

A method for providing optimal process parameters for a fluid bed granulation process may be executed using one or more components of a system such a system 100 shown in FIG. 1. An exemplary method 400 for providing optimal process parameters for a fluid bed granulation process is shown in FIG. 4.

Method 400 may begin with the receipt of a plurality of intrinsic properties of an input powder to be granulated (step 402). The plurality of intrinsic properties may be provided by a user via a user interface (e.g., UI 102) and may be received by one or more processors of a computing system (e.g., computing system 104). The plurality of intrinsic properties of the input powder may include physical properties of the input powder that may affect the granulation of the input powder, for example the bulk density of the input powder or a threshold temperature value or a temperature range associated with degradation of an attribute (e.g., the efficacy and/or safety) of the input powder.

In addition to the intrinsic properties of the input powder, granulation requirements for granules formed from the input powder during the fluid bed granulation process (step 404) and a plurality of fixed properties of the fluid bed granulation system to be used to execute the granulation process (step 406) may be received. Like the input powder properties, the granulation requirements and the fixed properties of the fluid bed granulation system may be provided by a user via a user interface. The granulation requirements may include desired granule properties such as a desired granule size distribution and a required granule density, while the fixed properties of the granulation system may comprise include a maximum rate at which the granulation system can spray liquid binder and a maximum volume of input powder that the granulation system can granulate during a given granulation session.

Using the received information, the granulation of the input powder in the fluid bed granulation system may be thermodynamically modeled (step 408). The computing system may thermodynamically model the granulation of the input powder by applying thermodynamic principles that govern the physical and chemical behavior of moist air to model conditions within the fluid bed granulation system during the granulation process. The information received in steps 402-406 may allow the computing system to determine the values of various thermodynamic quantities at various locations within the granulation system or at various stages of the granulation process which may, in turn, allow the computing system to determine optimal values of the process parameters for the granulation system. The specific process parameters for which optimized values are determined may depend on the particular make and model of the granulation system being used for the granulation process. Once optimized values of the process parameters have been determined, said values may be provided to the user on the user interface (step 410).

### Thermodynamic Model

As described, the optimal adjustable parameters for the fluid bed granulation system may be determined by thermodynamically modeling granulation of the input powder in the fluid bed granulation system. The computing system may be configured to use thermodynamic relations that govern the behavior of moist air to analyze thermodynamic conditions in the fluid bed granulation system and to determine the values of various thermodynamic properties (e.g., temperature, humidity, etc.) at various locations within the fluid bed granulation system or at various stages of the fluid bed granulation process. The optimal adjustable parameters for the fluid bed granulation system may be determined using the thermodynamic properties provided by the thermodynamic model.

### Definitions

Thermodynamic quantities used to thermodynamically model granulation of an input powder can include one or more of the following:
Barometric pressure (p): the pressure within Earth's atmosphere; dependent on altitude.
Dry Air (d.a.): Air that contains (or is assumed to contain) no water vapor.
Moist Air (m.a.): Binary mixture of dry air and water vapor.
Saturated Air (s.a.): An equilibrium state between moist air and condensed water. At a given temperature, saturated air contains the maximum possible amount of water vapor.
Universal Gas Constant (R=831.41 J/(kg·mol·K)): Proportionality constant that relates energy scale to scales of temperature and substance amount.
Gas Constant, Dry Air (R_{d.a.}=287.055 J/(kg·mol· K)): Specific gas constant for dry air.
Gas Constant, Water Vapor (R_{w}=461.50 J/(kg·mol·K)): Specific gas constant for water vapor.
Vapor Pressure of Water in Saturated Moist Air (pₛ): Pressure exerted by water vapor at a given temperature T.
Saturation Pressure of Water Vapor (p_{ws}): Pressure of water vapor in the absence of air at a given temperature T. In some examples, p_{ws} may differ negligibly from pₛ.
Partial Pressure of Dry Air (p_{d.a.}): The pressure of dry air in a given volume at a given temperature, assuming the dry air alone occupied the entire volume.
Partial Pressure of Water Vapor (p_{w}): The pressure of water vapor in a given volume at a given temperature, assuming the water vapor alone occupied the entire volume.
Pressure of Moist Air (p): Sum of the partial pressure of dry air and the partial pressure of water vapor in a moist air sample.
Mole Fraction of Water Vapor (x_{w}): The amount of water vapor (expressed in moles) in a sample divided by the total amount of all constituents in the sample; may be defined in terms of the partial pressures of dry air and water vapor in the sample.
Mole Fraction of Dry Air (x_{d.a.}): The amount of dry air (expressed in moles) in a sample divided by the total amount of all constituents in the sample; may be defined in terms of the partial pressures of dry air and water vapor in the sample.
Humidity Ratio of Moist Air (W): Ratio of the mass of water vapor (M_{w}) to the mass of dry air (M_{w}) contained in a sample of moist air; may be defined in terms of the mole fractions of water vapor and dry air along with the molecular masses of water vapor (m_{w}) and dry air (m_{d.a.}) contained in the sample.
Saturation Humidity Ratio (Wₛ(t, p)): Humidity ratio of moist air saturated with respect to water (or ice) at a given temperature t (in Celsius) and a given pressure p.
Specific Humidity (γ): Ratio of the mass of water vapor in a moist air sample to the total mass of the moist air sample.
Specific Volume of Moist Air (v): Total volume of humid air per mass unit of dry air in a moist air sample.
Density of Moist Air (ρ): Ratio of the total mass of moist air to the total volume (V) of moist air.
Absolute Humidity (d): Ratio of the mass of water vapor in a sample to the total volume (V) of the sample.
Specific Volume of Dry Air (v_{d.a.}): Volume (in m³) occupied by one kilogram of dry air.
Degree of Saturation (μ): Ratio of the humidity ratio (W) to the humidity ratio of saturated moist air (Wₛ) at the same temperature and pressure.
Relative Humidity (ϕ): Ratio of the mole fraction of water vapor in a moist air sample to the mole fraction of saturated air in an air sample saturated at the same temperature and pressure.
Dry-Bulb Temperature (t^{db}): Air temperature measured by a thermometer freely exposed to the air but shielded from radiation and moisture.
Specific Enthalpy of Dry Air at t^{db} (h_{d.a.}): Enthalpy per unit mass of dry air in a sample.
Specific Enthalpy of Saturated Water Vapor at t^{db} (h_{g.}): Enthalpy per unit mass of saturated water vapor in a sample.
Specific Enthalpy of Moist Air at t^{db} and p (h): Enthalpy per unit mass of moist air in a sample.
Specific Enthalpy of Condensed Water (h_{w}): Enthalpy per unit mass of condensed (liquid or solid) water in equilibrium with saturated moist air at a specified temperature and pressure.
Specific Enthalpy of Moist Air at Saturation (hₛ^{wb}): Enthalpy per unit mass of saturated moist air in a sample.
Dew Point (t^{dp}): Temperature to which air must be cooled in order to become saturated with water vapor at a given air pressure and humidity ratio.
Wet-Bulb Temperature (t^{wb}): Temperature at which water (liquid or solid) can adiabatically bring air to saturation by evaporating at a given dry-bulb temperature and a given humidity ratio.
Drying Capacity (DC): Amount of water that moist air can potentially remove from a system per unit volume if it leaves the system at 100% relative humidity; may depend on the absolute humidity of the moist air at the dry-bulb and wet-bulb temperatures.
Moisture Removal Capacity (\dot{H}): Amount of water that the moist air, per unit time at a certain air flow, can potentially remove from a system if it leaves the system at 100% relative humidity; may depend on the drying capacity and the flow rate of the moist air.
Drying Rate (DR): Amount of water per unit volume that the moist air will remove from a system if it exits the system at a temperature greater than its wet-bulb temperature; may depend on the absolute humidity of the moist air upon introduction to and exiting from the system.
Moisture Removal Rate ( [symbol] ): Amount of water that the moist air, per unit time at a certain air flow, will remove from a system if it exits the system at a temperature greater than its wet-bulb temperature; may depend on the drying rate and the flow of moist air.
Spray Rate (SR): Rate at which aqueous liquid is introduced into a system.
Moisture Addition Rate ( [symbol] ): Rate at which liquid water is introduced into a system; may depend on the water content (r_{w}) of an aqueous solution being introduced to the system.
Flow Rate of Moist Air (\dot{V}): Rate at which moist air flows through the system.

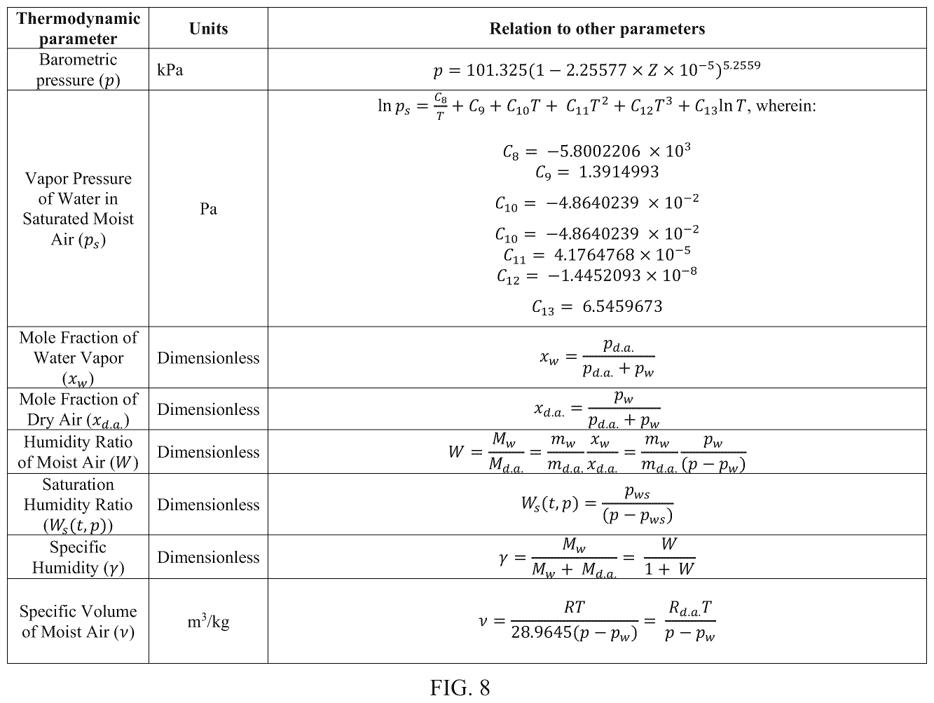

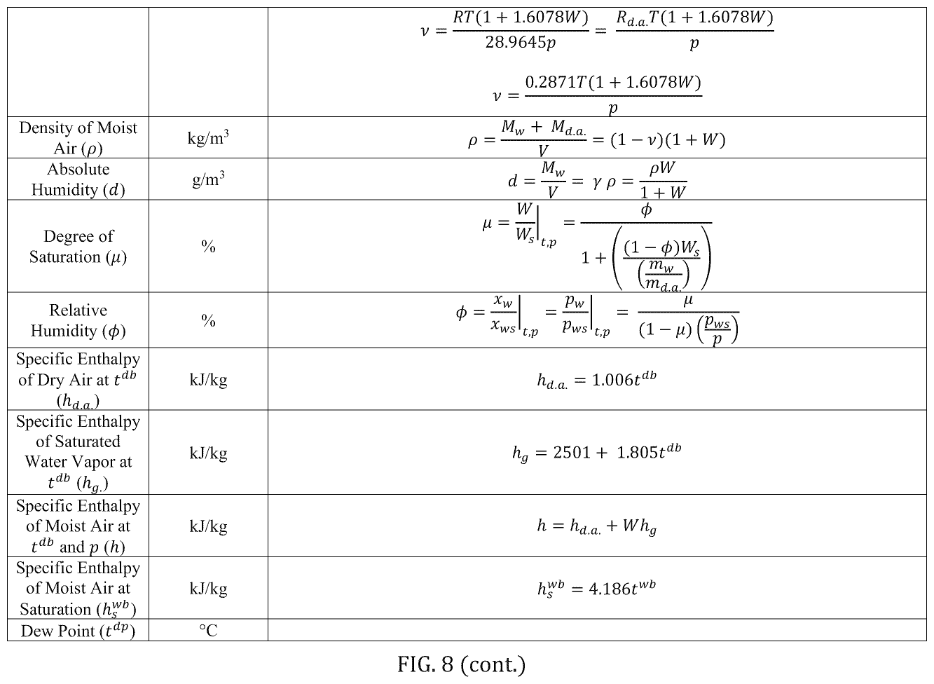

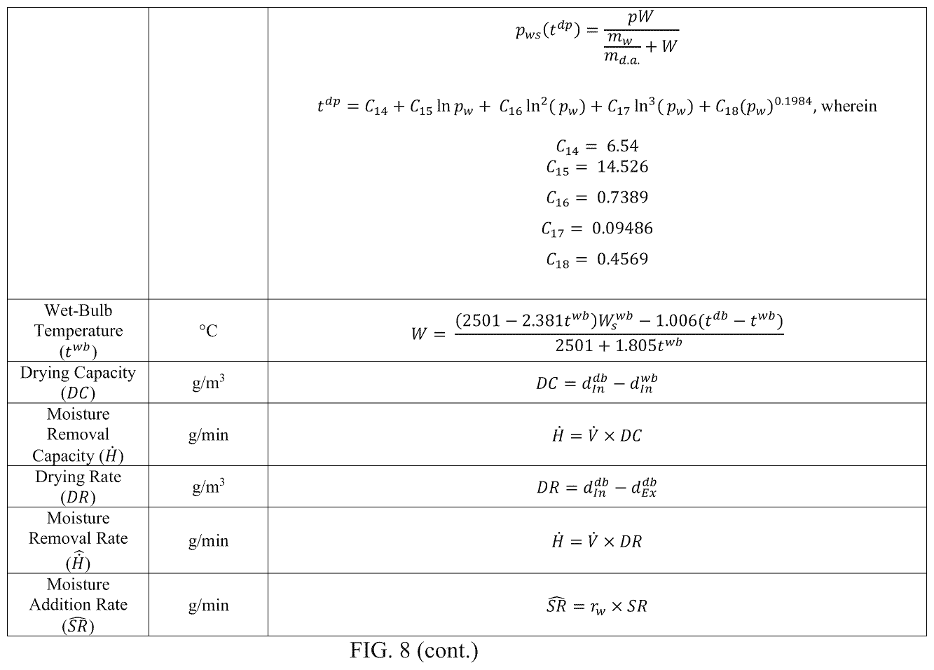

Exemplary relationships between the quantities defined above can be found in FIG. 8. These examples are provided for illustrative purposes; alternative thermodynamic relationships may be used depending on the information that is available for a specific fluid bed granulation process.

### Example

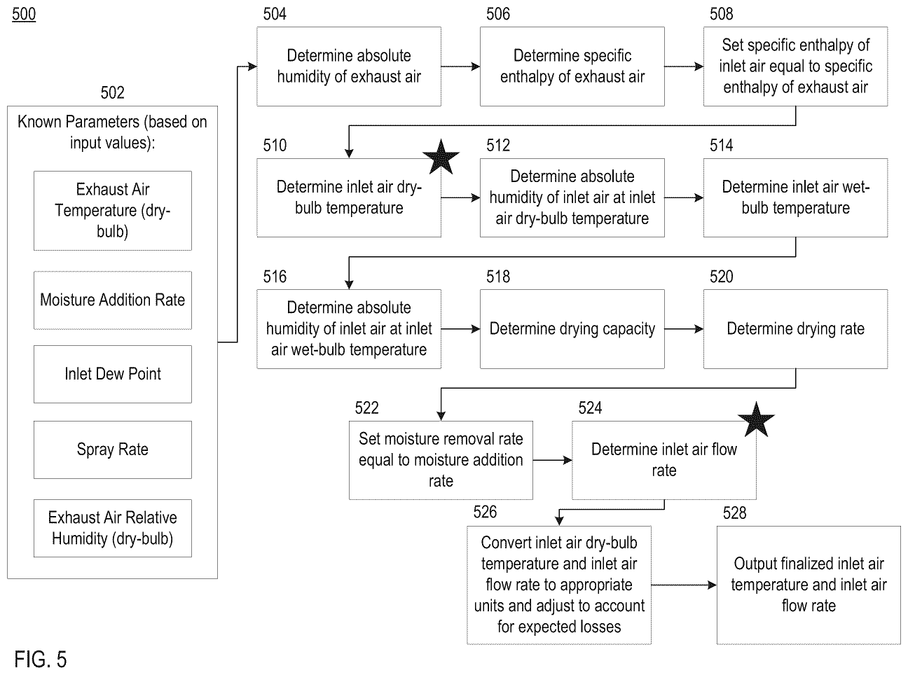

An example method 500 for thermodynamically modeling granulation of an input powder in a fluid bed granulation system is shown in FIG. 5. A computing system (e.g., computing system 104 shown in FIG. 1) may be configured to automatically (i.e., algorithmically) execute one or more steps of method 500 upon receipt of relevant information about the fluid bed granulation process to be performed.

First, known thermodynamic parameters may be determined based on the information provided by the user (step 502). The known parameters may include values directly provided by the user (e.g., batch size, granulation system capabilities, e.g., volume capacity, inlet air flow range, inlet air flow dew point range, and spray rate range) or may be deduced based on the provided information, for example by making certain assumptions about the thermodynamic conditions within the granulation system. These assumptions may include, for instance, assuming that the thermodynamic conditions within the granulation system can be accurately approximated using ideal gas laws or by assuming that heat loss from the granulation system during the granulation process is negligible. Such assumptions may allow thermodynamic parameters such as the dry-bulb air temperature at the exhaust of the granulation system to be determined by, e.g., equating the dry-bulb air temperature at the exhaust to a temperature value at or below a threshold temperature associated with degradation of the input powder. Other thermodynamic parameters that may be determined based on information provided by the user include the relative humidity of the exhaust air (which may be determined based on a final granule bulk density and a particle size distribution) and spray rate and inlet air flow properties (which may be determined based on the batch size and the capabilities of the granulation system being used). In some embodiments, manufacturing concerns (e.g., process time) can determine particular combinations of known thermodynamic parameters that are optimal for a specific product.

Using the known thermodynamic parameters, values of one or more thermodynamic properties may be determined (steps 504-524). The specific parameters indicated in steps 504-524 are intended only as examples and should not be construed as limiting the disclosure. The properties whose values are determined during the thermodynamic modeling of the granulation process may depend upon the particular process parameters being optimized, which may in turn depend upon the fluid bed granulation system being used to execute the granulation process.

A first thermodynamic parameter that may be determined (step 504) is the absolute humidity of air exiting the exhaust (e.g., exhaust 234) of the fluid bed granulation system. The absolute humidity of air exiting the exhaust may depend upon known thermodynamic parameters such as the dry-bulb air temperature at the exhaust and the relative humidity at the exhaust. Depending on the information provided by the user and the resulting known thermodynamic parameters, the absolute humidity of the exhaust air may be determined based on the following relations:

$$d = \frac{{M}_{w}}{V} = γ ρ = \frac{ρ W}{1 + W} \qquad ( 1 )$$

A second thermodynamic parameter that may be determined (step 506) is the specific enthalpy of the exhaust air. The specific enthalpy of the exhaust air may depend on the dry-bulb air temperature at the exhaust and the relative humidity at the exhaust or the absolute humidity of air exiting the exhaust, and can be determined using the following equation:

$$h = {h}_{d . a .} + W {h}_{g} \qquad ( 2 )$$

A third thermodynamic parameter that may be determined (step 508) is the specific enthalpy of air flowing into the inlet (e.g., inlet 222 shown in FIG. 2). The specific enthalpy of air flowing into the inlet may be determined by imposing a constraint requiring no enthalpy loss during the granulation process, i.e., by equating the specific enthalpy of the exhaust air to the specific enthalpy of air flowing into the inlet.

A fourth thermodynamic parameter that may be determined (step 510) is the dry-bulb air temperature at the inlet. Since the specific enthalpy of the inlet air depends upon the dry-bulb air temperature at the inlet, Equation 2 may be used to determine the dry-bulb air temperature at the inlet. The dry-bulb air temperature at the inlet may be one of the process parameters for the fluid bed granulation system; thus, the value determined in 510 may be one of the optimal process parameters.

A fifth thermodynamic parameter that may be determined (step 512) is the absolute humidity of inlet air at the inlet air dry-bulb temperature. The absolute humidity of inlet air may depend upon the dry-bulb air temperature at the inlet and the inlet dew point, and can, in some embodiments, be determined using Equation 1.

A sixth thermodynamic parameter that may be determined (step 514) is the wet-bulb temperature of the inlet air. The wet-bulb temperature of the inlet air may depend upon the inlet dew point, and the inlet air dry-bulb temperature, and can be determined using the following equation:

$$W = \frac{( 2501 - 2.381 {t}^{w b} ) {W}_{s}^{w b} - 1.006 ( {t}^{d b} - {t}^{w b} )}{2501 + 1.805 {t}^{w b}} \qquad ( 3 )$$

A seventh thermodynamic parameter that may be determined (step 516) is the absolute humidity of the inlet air at the wet-bulb temperature of the inlet air. The absolute humidity of the inlet air at the wet-bulb temperature may depend upon the wet-bulb temperature and can be determined using Equation 1.

An eighth thermodynamic parameter that may be determined (step 518) is the drying capacity of the granulation system. The drying capacity may depend upon the absolute humidity of the inlet air at the dry-bulb and wet-bulb temperatures, and can be determined using the following equation:

$$D C = {d}_{Inlet}^{d b} - {d}_{Inlet}^{w b} \qquad ( 4 )$$

A ninth thermodynamic parameter that may be determined (step 520) is the drying rate of the granulation system. The drying rate may depend upon the absolute humidity of the exhaust air and the inlet air at the dry-bulb temperatures, and can be determined using the following equation:

$$D R = {d}_{Inlet}^{d b} - {d}_{E xhaust}^{d b} \qquad ( 5 )$$

A tenth thermodynamic parameter that may be determined (step 522) is the moisture removal rate. Assuming moisture is added and removed from the system at the same rate, the moisture removal rate may be equated to the moisture addition rate, which may, in turn, have been determined based on the spray rate of the granulation system and the water content of the binder solution.

An eleventh thermodynamic parameter that may be determined (step 524) is the flow rate of air through the inlet. The flow rate of air through the inlet may be related to the moisture removal rate and the absolute humidity of the inlet and exhaust air at the dry-bulb temperatures, and can be determined using the following equation:

$$\hat{\dot{H}} = \dot{V} ( {d}_{Inlet}^{d b} - {d}_{Exhaust}^{d b} ) = \dot{V} \times D R \qquad ( 6 )$$

The flow rate of air through the inlet may be one of the process parameters for the fluid bed granulation system. The value determined in step 524 may, therefore, be one of the optimal process parameters.

After the dry-bulb temperature of the inlet air and the inlet air flow rate have been determined, their values may be adjusted as necessary (step 526), for example to express the values in units that the fluid bed granulation system accepts, or to account for predicted losses (e.g., of heat) that were previously ignored during method 500. Information about the predicted losses and the units accepted by the granulation system may be provided by the user via the user interface. The finalized optimal process parameters may then be output to the user interface (step 528).

### UI Features

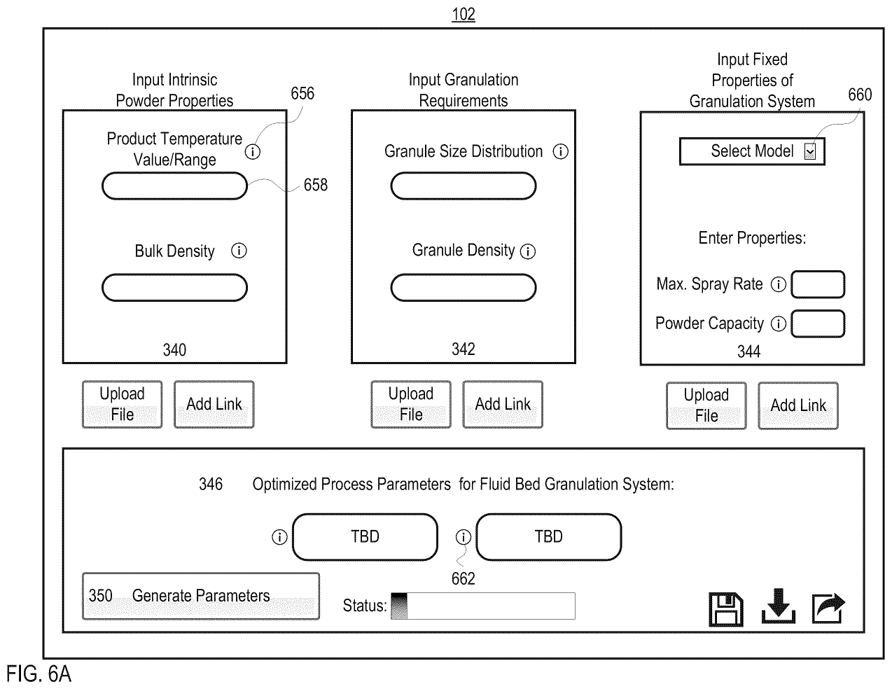

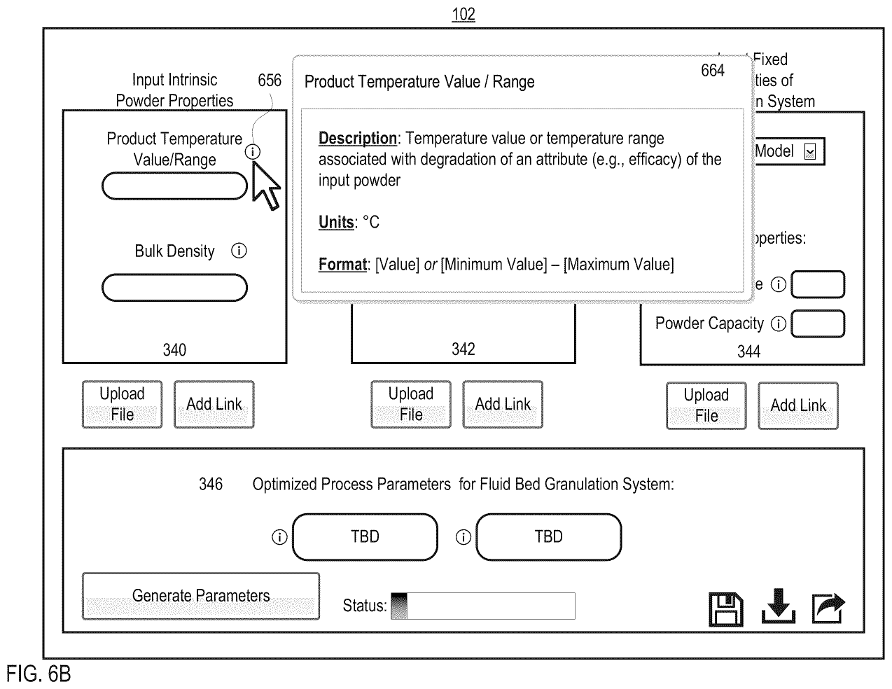

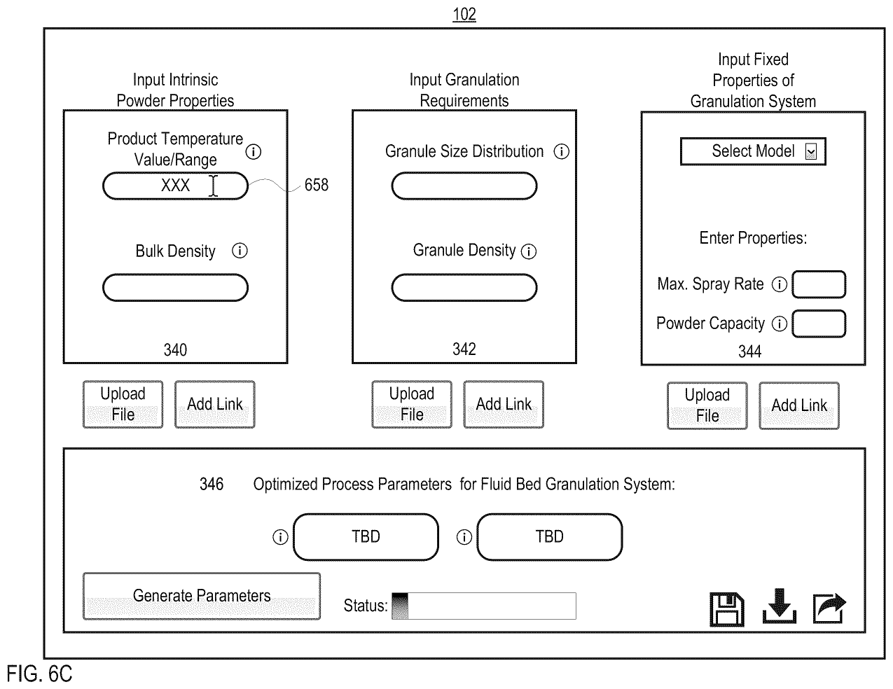

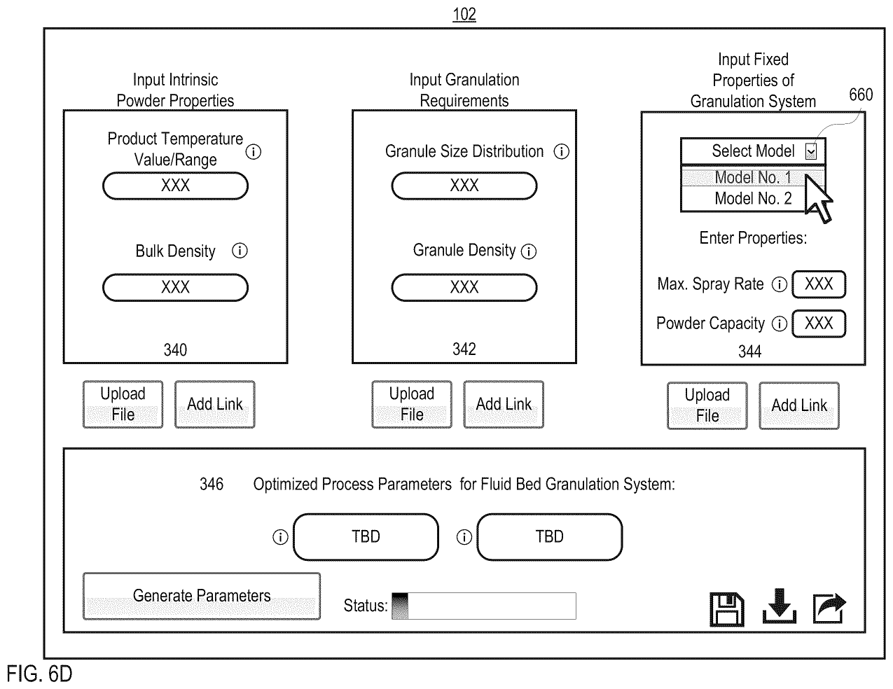

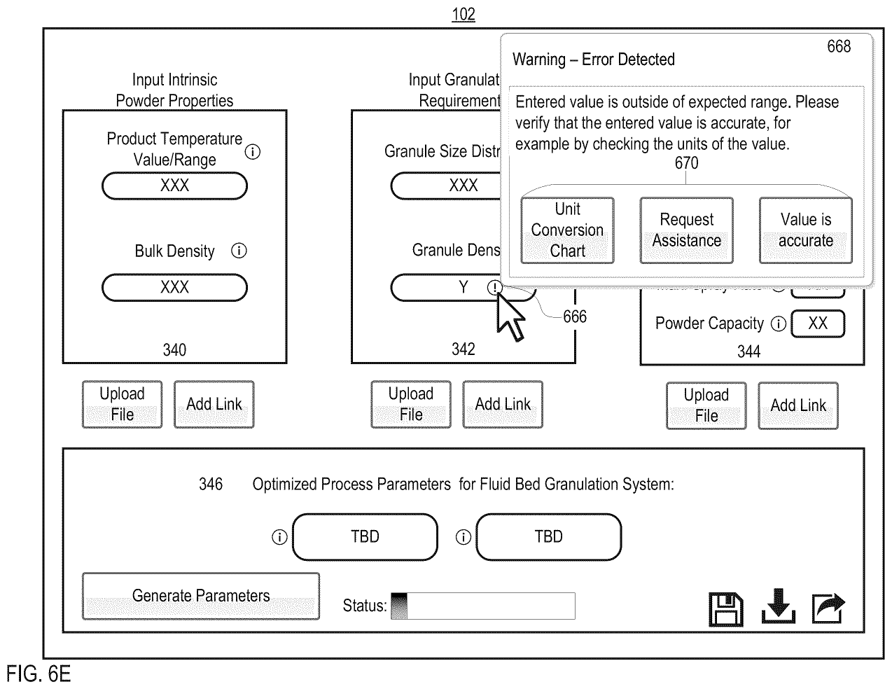

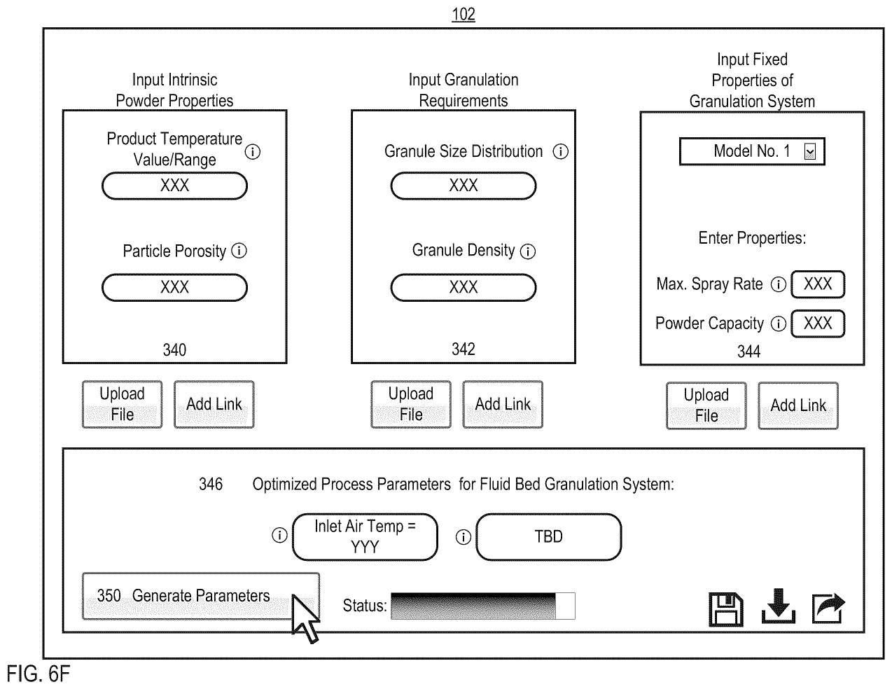

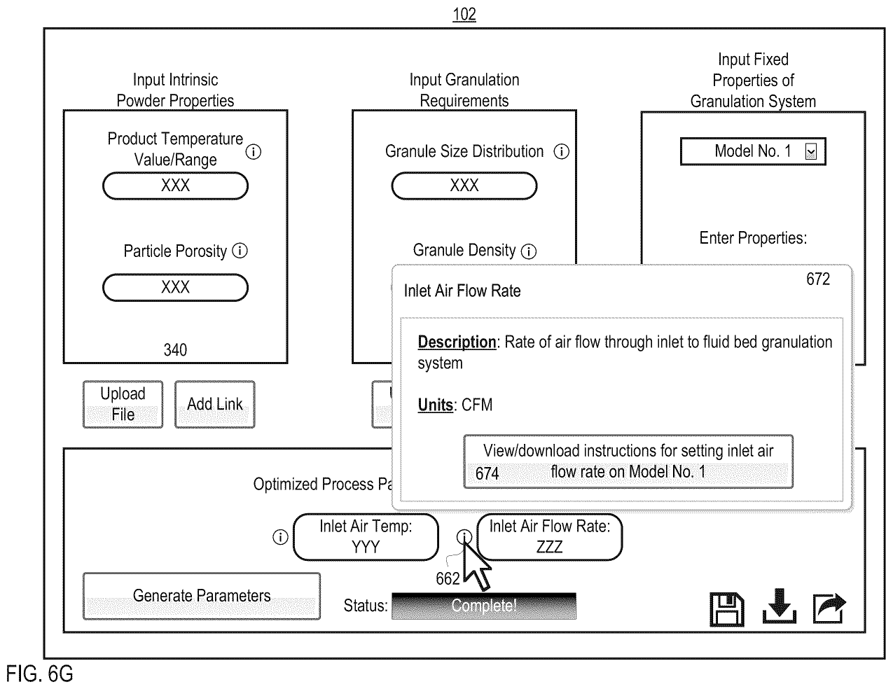

As previously discussed, user interface 102 for providing optimal adjustable parameters for a fluid bed granulation process may include a variety of features configured to assist the user as they provide the necessary information and receive the optimal parameters. Examples of such features are shown in FIGS. 6A-6G.

As shown, user input fields 340-344 may be configured to receive data in a variety of formats. Some user input fields, for example, may include text fields 658 configured to allow the user to input (e.g. by typing, as shown in FIG. 6C) known values (e.g., temperature values corresponding to the input powder's temperature tolerance). Other user input fields may include drop-down menus configured to allow the user to select relevant information from a stored list (e.g., menu 660 shown in FIG. 6D).

If the user requires additional description of a particular quantity requested in an input field, UI 102 may be configured to display an input information window 664 upon user request, e.g., if the user selects or hovers over an information icon 656 with their cursor (FIG. 6B). If user inputs a value that the computing system determines may be incorrect, UI 102 may be configured to display a warning icon 666 that flags the error (FIG. 6E). When the user hovers over or selects warning icon 666, UI 102 may be configured to display information about the detected error in an error window 668. Error window 668 may include user controls 670 configured to, for example, allow users to view a unit conversion chart, request assistance from an expert, or override the warning and proceed with the input as entered.

To cause UI 102 to provide the optimal adjustable parameters, users may select a user output control 350 configured to prompt the computing system to determine the optimal adjustable parameters (FIG. 6F). Once the optimal parameters are provided, UI 102 may be configured to display an output information window 672 upon user request, e.g., if the user selects or hovers over an information icon 662 with their cursor (FIG. 6G). Information window 672 may provide description of the optimal parameter, along with a link 674 to instructions for setting the value of the adjustable parameter for the granulation system to the optimal value.

### Fluid Bed Granulation Using Optimal Process Parameters

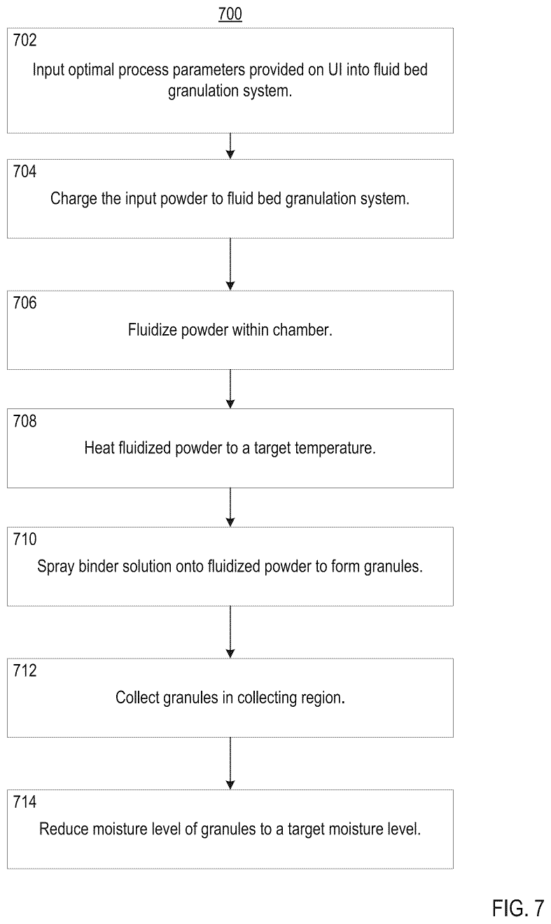

Once the optimal process parameters for the fluid bed granulation system have been determined, the fluid bed granulation process may be initiated. An exemplary method 700 for granulating an input powder using the optimal process parameters provided by the described systems and methods is shown in FIG. 7. After receiving the optimal process parameters determined by the computing system (e.g., via the UI), the operator of the fluid bed granulation system may input the optimal process parameters into the fluid bed granulation system (step 702). The input powder may then be charged to the fluid bed granulation system (step 704).

The fluid bed granulation system may supply conditioned air through an inlet to fluidize particles of the input powder within a chamber of the fluid bed granulation system (step 706). The fluidized powder may then be heated to a target temperature (step 708). The values of the temperature and the flow rate of the conditioned air that is supplied through the inlet may have been among the optimal process parameters set by the operator in step 702. The computing system may have determined these values based on a thermodynamic model of the granulation of the input powder to ensure that, for example, the inlet air temperature is a temperature to which the input powder can be safely exposed.

After the fluidized powder has reached the target temperature, the fluid bed granulation system may spray a binder solution into the fluidized powder (step 710). As the fluidized particles of powder moisten and collide, they may stick to one another and form granules. Once a pre-determined quantity of the binder solution is applied, the moisture level of the granules may then be reduced (e.g., by heating the granules to cause excess water content to evaporate) to a target moisture level (step 712). Once the granules have been sufficiently dried, they may be extracted from the fluid bed granulation system for further processing, for example for compression into pharmaceutical tablets.

The foregoing description, for the purpose of explanation, has been described with reference to specific embodiments and/or examples. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

Any of the systems, methods, techniques, and/or features disclosed herein may be combined, in whole or in part, with any other systems, methods, techniques, and/or features disclosed herein.

1. A method for providing optimized process parameters for a fluid bed granulation process, the method comprising:
receiving from a user a plurality of intrinsic properties of an input powder, wherein the plurality of intrinsic properties comprises a bulk density of particles of the input powder and a threshold temperature value or a temperature range associated with degradation of an attribute of the input powder;
receiving from the user granulation requirements for granules formed from the input powder during the fluid bed granulation process, wherein the granulation requirements comprise a granule size distribution and a required granule density;
receiving from the user a plurality of operational capabilities of a fluid bed granulation system, wherein the plurality of operational capabilities of the fluid bed granulation system comprises a minimum and a maximum volume capacity, a spray rate range, an inlet air flow range, an inlet air temperature range, and an inlet air dew point range;
thermodynamically modeling, using the plurality of intrinsic properties of the input powder, the granulation requirements, and the plurality of operational capabilities of the fluid bed granulation system, granulation of the input powder in the fluid bed granulation system to determine optimal process parameters for the fluid bed granulation system, wherein the optimal process parameters comprise an inlet air temperature and an inlet air flow rate of air supplied into an inlet of the fluid bed granulation system; and
providing the optimal process parameters for the fluid bed granulation system on a user interface.

2. The method of claim 1, wherein thermodynamically modeling granulation of the input powder in the fluid bed granulation system comprises determining an absolute humidity of exhaust air expelled by the fluid bed granulation system using the threshold temperature value or the temperature range associated with degradation of the attribute of the input powder.

3. The method of claim 2, wherein thermodynamically modeling granulation of the input powder in the fluid bed granulation system comprises determining a specific enthalpy of exhaust air expelled by the fluid bed granulation system using the threshold temperature value or the temperature range associated with degradation of the attribute of the input powder and the absolute humidity of exhaust air.

4. The method of claim 3, wherein thermodynamically modeling granulation of the input powder in the fluid bed granulation system comprises determining a specific enthalpy air at the inlet to the fluid bed granulation system based on the specific enthalpy of exhaust air expelled by the fluid bed granulation system.

5. The method of claim 4, wherein the inlet air temperature is determined based on the specific enthalpy air at the inlet to the fluid bed granulation system.

6. The method of claim 5, wherein thermodynamically modeling granulation of the input powder in the fluid bed granulation system comprises determining an absolute humidity of air at the inlet to the fluid bed granulation system using the inlet air temperature.

7. The method of claim 6, wherein thermodynamically modeling granulation of the input powder in the fluid bed granulation system comprises determining a wet-bulb temperature of air at the inlet to the fluid bed granulation system using the absolute humidity of air at the inlet to the fluid bed granulation system.

8. The method of claim 7, wherein thermodynamically modeling granulation of the input powder in the fluid bed granulation system comprises determining a drying capacity of the fluid bed granulation system and a drying rate of the fluid bed granulation system based on the absolute humidity of the inlet air at the dry-bulb temperature and at the wet-bulb temperature.

9. The method of claim 8, wherein the inlet air flow rate of air supplied into an inlet of the fluid bed granulation system is determined based on the drying capacity and the drying rate for the fluid bed granulation system.

10. The method of claim 1, comprising:
providing the optimal process parameters for the fluid bed granulation system to the fluid bed granulation system; and
granulating the input powder using the fluid bed granulation system.

11. A user interface for providing optimal process parameters for a fluid bed granulation process, the user interface comprising:
a first input field for receiving, from a user, a plurality of intrinsic properties of an input powder, wherein the plurality of intrinsic properties comprises a bulk density of particles of the input powder and a threshold temperature value or a temperature range associated with degradation of an attribute of the input powder;
a second input field for receiving, from the user, granulation requirements for granules formed from the input powder during the fluid bed granulation process, wherein the granulation requirements comprise a granule size distribution and a required granule density;
a third input field for receiving, from the user, a plurality of operational capabilities of a fluid bed granulation system, wherein the plurality of operational capabilities of the fluid bed granulation system comprises a minimum and a maximum volume capacity, a spray rate range, an inlet air flow range, an inlet air temperature range, and an inlet air dew point range;
an output field configured to provide optimal process parameters for the fluid bed granulation system, wherein the optimal process parameters are determined by thermodynamically modeling, using the plurality of intrinsic properties of the input powder, the granulation requirements, and the plurality of operational capabilities of the fluid bed granulation system, granulation of the input powder in the fluid bed granulation system, and wherein the optimal process parameters comprise an inlet air temperature and an inlet air flow rate for air supplied into an inlet of the fluid bed granulation system.

12. The user interface of claim 11, wherein the user interface is configured to display an information window comprising information associated with the first user input field, the second user input field, or the third user input field upon user request.

13. The user interface of claim 12, wherein the information associated with the first user input field, the second user input field, or the third user input field comprises information about a format of a requested user input or a description of a requested user input.

14. The user interface of claim 1, wherein the user interface is configured to display a warning window indicating that a possible error has been detected when the user provides a value to the first user input field, the second user input field, or the third user input field that is outside of a predetermined range.

15. The user interface of claim 1, wherein the output field provides the optimal process parameters by displaying values of the optimal process parameters.

16. The user interface of claim 1, wherein the output field provides the optimal process parameters by providing a downloadable file comprising the optimal process parameters.

17. The user interface of claim 1, wherein the user interface is configured to provide information about inputting the optimal process parameters into the fluid bed granulation system upon user request.

18. A system for providing optimal process parameters for a fluid bed granulation process, the system comprising:
a fluid bed granulation system;
a user interface comprising:
a first input field configured to receive, from a user, a plurality of intrinsic properties of an input powder, wherein the plurality of intrinsic properties comprises a bulk density of particles of the input powder and a threshold temperature value or a temperature range associated with degradation of an attribute of the input powder,
a second input field configured to receive, from the user, granulation requirements for granules formed from the input powder during the fluid bed granulation process, wherein the granulation requirements comprise a granule size distribution and a required granule density,
a third input field configured to receive, from the user, a plurality of operational capabilities of a fluid bed granulation system, wherein the plurality of operational capabilities of the fluid bed granulation system comprises a minimum and a maximum volume capacity, a spray rate range, an inlet air flow range, an inlet air temperature range, and an inlet air dew point range, and
an output field; and
a computing system comprising one or more memories and one or more processors configured to:
receive, from the user interface, the plurality of intrinsic properties of the input powder, the granulation requirements, and the plurality of operational capabilities of the fluid bed granulation system,
thermodynamically model, using the plurality of intrinsic properties of the input powder, the granulation requirements, and the plurality of operational capabilities of the fluid bed granulation system, granulation of the input powder in the fluid bed granulation system to determine optimal process parameters for the fluid bed granulation system, wherein the optimal process parameters comprise an inlet air temperature and an inlet air flow rate for air supplied into an inlet of the fluid bed granulation system, and
provide, using the output field of the user interface, the optimal process parameters for the fluid bed granulation system.
